(19) United States
(12) Patent Application Publication
(10) Pub. No.: US 2017/0281653 A1
(43) Pub. Date: Oct. 5, 2017

(54) DERMOCOSMETIC OR PHARMACEUTICAL USE OF A COMPOSITION CONTAINING AT LEAST ONE INHIBITOR OF CERTAIN CHEMOKINES

(71) Applicants: GREENPHARMA, ORLÉANS (FR); CENTRE NATIONAL DE LA RECHERCHE SCIENTIFIQUE (CNRS), PARIS CEDEX 16 (FR); UNIVERSITÉ DE STRASBOURG, STRASBOURG (FR)

(72) Inventors: Philippe BERNARD, La Ferté Saint-Aubin (FR); Dayana ABBOUD, IlIkirch (FR); Jean-Luc GALZI, Weitbruch (FR); Nelly FROSSARD, Strasbourg (FR); Christelle PILLARD, Orléans (FR); Quoc Tuan DO, Orléans (FR)

(21) Appl. No.: 15/514,227

(22) PCT Filed: Sep. 22, 2015
(86) PCT No.: PCT/FR15/00185

§ 371 Date: Mar. 24, 2017

(30) Foreign Application Priority Data
Sep. 24, 2014 (FR) 14/02163

(51) Int. Cl. A61K 31/618; A61K 45/06; A61K 9/14; A61K 9/00; A61K 9/06

(52) U.S. Cl. CPC A61K 31/618; A61K 9/0014; A61K 9/06; A61K 9/14; A61K 45/06

(57) ABSTRACT

The present invention relates to the cosmetic or pharmaceutical use for a composition comprising a salicylic or nicotinic acid derivative inhibiting chemokines, for preventing or treating chronic internal and/or external inflammatory conditions.

The present invention relates to the use of some derivatives inhibiting chemo-attractant cytokines (generally called chemokines), in cosmetic or pharmaceutical compositions. More precisely, the present invention relates to new applications of said derivatives of salicylic or nicotinic acid for treating chronic inflammatory diseases; it is possible to mention in this regard, external chronic inflammatory diseases such as dermatoses (acne, atopic dermatitis, psoriasis, eczema, dry skin with atopic tendencies, skin rash . . . ) or internal such as Crohn's disease, asthma, allergic asthma and allergic rhinitis.

Crohn's disease is a chronic inflammatory bowel disease. The article of Grip O. and Janciauskiene S. (Atorvastatin reduces plasma levels of chemokine (CXCL10) in patients with Crohn's disease, PloS One, 2009, 4(5): e5263) shows the importance of chemokines in this pathology.

Allergic rhinitis is a chronic disease often linked to chronic dermatoses. An article of Takeuchi S. et al. (Changes in thymus- and activation-regulated chemokine (IARC) associated with allergen immunotherapy in patients with perennial allergic rhinitis. J Investig Allergol Clin Immunol, 2005, 15(3), 172-6) shows the involvement of chemokines in this pathology.

Dermatoses are skin and mucous membranes disorders that are characterized by unsightly manifestations such as red patches and desquamation zones. Several pathologies are grouped under the name of dermatoses. It is possible to mention, as non-limiting examples, eczema, atopic dermatitis, psoriasis, seborrheic dermatitis or acne. These dermatoses are very often the result of inflammatory phenomena and immune disorders.

In the case of atopic dermatitis, lesions are characterized by a severe dryness of the skin and by inflammatory lesions: papular, vesicular, squamous and very pruritic erythematous eruptions (itching). From a histological point of view, atopic dermatitis is characterized, as many dermatoses, by an infiltration of lymphocytes, monocytes and polynuclear eosinophils around the small vessels and the capillaries. From a biochemical point of view, it has been shown that chemokines are strongly involved in dermatoses and in chronic inflammatory diseases in general. For example, the article of Folsgaard et al. (Cord blood Th-2-related chemokine CCL22 levels associate with elevated total-IgE during preschool age, Clin. Exp. Allergy, 2012, 42(11), 1596-603) highlights the importance of the CCL22 chemokine in atopic dermatitis. Another article of Hashimoto et al. (Macrophage-derived chemokine (MDC)/CCL22 produced by monocyte derived dendritic cells reflects the disease activity in patients with atopic dermatitis, Journal of Dermatological Science, 2006, 44(2), 93-9) highlights the importance of the same CCL22 chemokine in skin diseases.

Chemokines are a family of small, mostly soluble, proteins of 8-14 kD. Their most studied function is the attraction and control of the activation state of cells of the immune system. Some chemokines have proven to be good therapeutic targets for treating internal and external chronic inflammatory diseases in humans or animals.

The present invention shows new cosmetic, pharmaceutical and veterinary applications of some agents effective for directly inhibiting chemokines and treating chronic inflammatory diseases in human or animals. In this patent application, the generic term “pharmaceutical” is used for defining a product intended for use in humans as well as a product intended for use in animals.

The invention relates to the use of a dermocosmetic or pharmaceutical composition for treating inflammatory diseases of the human or animal skin, characterized in that said composition contains, in a pharmaceutically acceptable vehicle, an effective amount of at least one active agent having the general formula (I):

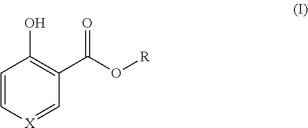

wherein:
X represents a —CH— group or a nitrogen atom;
R represents a (C₁-C₆) alkyl, a (C₃-C₆) cycloalkyl group, wherein one or more —CH₂— groups may be replaced by —O— or may be substituted by one or more radicals selected from (C₁-C₆) alkyl, hydroxy and alkoxy radicals, said compound of formula (I) may be in the form of a basic or acid addition salt.

When, in the composition implemented in the use according to the invention, the active agent is in the form of an acid addition salt, said acid is advantageously selected from the group consisting of hydrochloric acid, hydrobromic acid, sulfuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, methane- or ethane-sulfonic acid and camphoric acid.

When, in the composition implemented in the use according to the invention, the active agent is in the form of a basic addition salt, said base is advantageously selected from the group consisting of sodium or potassium hydroxide, triethylamine and tert-butylamine.

The compounds of formula (I) may possess one or more asymmetric centers and may then be isolated in an optically active form or in the form of their racemic mixture. The methods allowing to obtain the optically active forms, for example by resolution of a racemic form or by synthesis using racemic starting products, are well known to one of skill in the art.

For the definition of the compounds of formula (I), the term “alkyl” refers to a linear or branched hydrocarbon radical advantageously having from 1 to 6 carbon atoms, such as methyl, ethyl, propyl, isopropyl, butyl, isobutyl, tert-butyl, pentyl, neopentyl, n-hexyl. The alkyl groups may be substituted by one or more hydroxyl groups and/or one or more alkoxy groups as defined hereafter.

The term “cycloalkyl” refers to a cyclic hydrocarbon system, comprising from 3 to 6 carbon atoms and that may be mono- or poly-cyclic. It is possible to mention in particular cyclopropyl and cyclohexyl radicals.

The “alkoxy” groups correspond to the linear or branched alkyl groups defined above linked via an —O— (ether) bond. The methoxy, ethoxy, n-propyloxy, i-propyloxy, n-butoxy, s-butoxy, t-butoxy, n-pentoxy and s-pentoxy groups are particularly preferred. The alkoxy groups may be substituted by an alkyl group as defined above or by another alkoxy group.

Among the compounds of formula (I) which may be used as active agent in the compositions implemented in the use according to the invention, it is possible to particularly mention:
2,3-dihydroxypropyl 2-hydroxybenzoate (coded GPN000136)
(S)-2,3-dihydroxypropyl 2-hydroxybenzoate (referenced GPS008506)
(R)-2,3-dihydroxypropyl 2-hydroxybenzoate (referenced GPS008505)
2,3-dihydroxypropyl 4-hydroxynicotinate and its isomers
dipropylene glycol salicylate and its isomers
propylene glycol salicylate and its isomers.

The invention relates in particular to a use that implements a composition comprising at least one compound of formula (I) as active agent for treating inflammatory diseases of the skin. Such a composition may also comprise at least one active agent different from those of formula (I) for exerting at least one complementary or synergistic action.

According to another aspect of the invention, the implemented composition according to the invention is in the form of capsules, a cream, a gel, a lotion, a milk, an emulsion, a biphasic emulsion, an O/W or W/O emulsion, a triphasic emulsion, a solution, an ointment, a body oil, a shampoo, a soap, a lip protection stick, a stick and a pencil for makeup, a mask, a nanocapsule, a liposome, transdermic patches for topical applications.

In the form of a gel, the composition implemented according to the invention may comprise suitable excipients, such as cellulose esters, or other gelling agents, such as the carboxylic polymer “Carbopol®” and guar gum, for example.

In the form of emulsions, the implemented compositions according to the invention have a good stability and may be stored for a required period of time for the use at temperatures between 0 and 50° C., without the constituents sedimenting or the phases separating.

According to another aspect of the invention, in the composition implemented according to the invention, the active agent(s) is (are) placed in encapsulation means selected from the group consisting of microspheres, liposomes, glycospheres, chylomicrons, macro-, micro- and nanoparticles, macro-, micro- and nanocapsules.

According to another aspect of the invention, in the implemented composition according to the invention, the active agent(s) is (are) absorbed or adsorbed on powdery organic polymers, talcs, bentonite or other powdered mineral supports.

According to another aspect of the invention, in the composition implemented according to the invention, the content in active agent(s) of formula (I) is from 0.01% to 5% by weight with respect to the total weight of the composition.

According to another aspect of the invention, in the composition implemented according to the invention, the active agent(s) of formula (I) is (are) in encapsulated form and constitute(s) from 0.5 to 5% by weight with respect to the total weight of the composition.

The invention notably relates to the use of a composition as previously described for preventing and/or treating chronic inflammatory diseases whether internal, such as Crohn's disease, asthma, allergic asthma and allergic rhinitis, or external linked to seborrheic, acne, inflammatory and immunological activities; such a composition may in particular be used for preventing and/or treating dermatoses, psoriasis, atopic dermatitis, as well as inflammatory conditions of the skin and hair systems causing chronic itching, irritation, redness or functional disorders of the capillary fragility.

The invention has thus shown the possibility of using the compounds of formula (I) as active agent(s). These compounds have very good properties for inhibiting some chemokines, such as CCL22 and/or CCL17.

The present invention therefore precisely aims at providing a new pharmaceutical, dermatological or cosmetic use implementing compositions which can treat internal and external chronic inflammatory diseases such as irritated, injured or sensitized skin by external or internal aggressions, in general, and in particular, dermatoses.

Thus, the present invention relates to a cosmetic use of at least one compound of formula (I) for inhibiting the CCL22 and/or CCL17 chemokine, for soothing dry skin with atopic tendencies and/or for improving and/or enhancing skin hydration. It therefore relates to uses for treating chronic inflammatory pathologies such as Crohn's disease, allergic rhinitis, asthma, dermatoses such as atopic dermatitis, eczema and psoriasis.

According to the therapeutic goal of the invention, the derivatives of formula (I) which can inhibit the CCL22 and/or CCL17 chemokine may be administered by the topical route but they may also be administered by the oral route. These derivatives may be used as such, in a liquid or powder form, purified or not.

The derivatives of formula (I) which can inhibit chemokines may, in the implemented compositions, be associated with each other or with other compounds which complement the effect or even synergize this effect. The protective activity of the derivatives of formula (I) with respect to free radicals and UV-rays is interesting in the capillary field, in particular in the case of association with substances facilitating the good condition of the scalp and of the hair. It is possible to mention associations with mucopolysaccharides, minerals, vitamins, ceramides, vegetal oils, antiradical substances, UV filters, flower or fruit acids.

Similarly, the repairing activity of the compounds of formula (I) which can inhibit chemokines is particularly interesting when they are associated to substances having a healing effect such as proteins, hyaluronic acid, amino acids, or with anti-inflammatory, anti-aging, after-sun, anti-acne or anti-dermatosis substances.

As such, the compositions implemented according to the invention are particularly adapted to a topical use for preventing and/or treating many skin defects, in particular as repairing and/or protective agents of the skin and the hair system such as head hair, for fighting against external aggressions linked to pollution, sun, oxidative stress, aging and skin pathologies leading to a dysfunction of epidermal or hair homeostasis.

These cosmetic compositions implemented according to the invention may also be in the form of lotions or solutions wherein compounds of formula (I) are in an encapsulated form, for example in microspheres. For example, these microspheres may be made of fat bodies, agar and water. The active agents may also be incorporated in vectors such as liposomes, glycospheres, chylomicrons, macro-, micro-, nanoparticles as well as macro-, micro and nanocapsules and may also be adsorbed on powdery organic polymers, talcs, bentonites and other mineral supports.

For the manufacture of the compositions implemented according to the invention, compounds of formula (I) may be mixed with excipients generally used in cosmetics. The cosmetic compositions implemented according to the invention may therefore contain additives or adjuvants usual in cosmetology, as for example antibacterial agents or perfumes as well as extraction and/or synthesis lipids, gelling and viscosity polymers, surfactants, emulsifiers, hydro- or lipo-soluble active substances, plant extracts, tissue extracts, marine extracts, or synthesis active substances.

The dermo-cosmetic or pharmaceutical use of the compounds of formula (I) comprises all the body and skin care products, including sun products, for protection and tanning, anti-aging, anti-seborrheic, tonic products, products improving the appearance of the skin including the treatment of acne, the treatment of redness of the skin, the treatment of the scalp and that of hair loss.

The cosmetic compositions implemented according to the invention may also comprise other complementary active agents selected for their action, for example for sun protection, anti-wrinkle effect, antiradical and antioxidant activity, anti-irritant activity, cellular nutrition, cellular respiration, hydration and regeneration, anti-seborrheic treatments, as well as other active agents having an action on skin tonicity and hair protection.

The cosmetic compositions implemented according to the present invention are preferably to be used daily by applying them one or more times a day.

The cosmetic composition implemented according to the present invention are very well tolerated, they present no phototoxicity and their application to the skin, for extended periods of time, does not lead to any systemic effect.

The invention also relates to the use of compounds of formula (I) for the manufacture of pharmaceutical compositions having an anti-inflammatory and/or dermoprotective activity. These compositions are in particular useful for preventing and/or treating dermatologic diseases linked to seborrheic, acne, inflammatory and immunological activities.

In summary, the invention therefore relates to the use of a pharmaceutical, dermatological or cosmetic composition, containing compounds of formula (I) which can inhibit some chemokines, for preventing and/or treating the pathologies or disorders resulting from:

natural or premature aging of the skin or hair;
dermatoses, such as psoriasis, acne and atopic dermatitis;
inflammatory and/or immunity conditions of the skin and hair systems, causing chronic itching, irritations, redness, persistent irritations and itching, and functional disorders of the capillary fragility.

The present invention is now illustrated by the examples described hereafter.

Examples 1 to 3 illustrate a method for preparing compounds of formula (I) which can inhibit the CCL22 chemokine according to the invention.

The starting products are commercially available or may be synthesized by classic methods known to one of ordinary skill in the art.

### EXAMPLE 1

### Synthesis of 2,3-dihydroxypropyl 2-hydroxybenzoate (Coded GPN136)

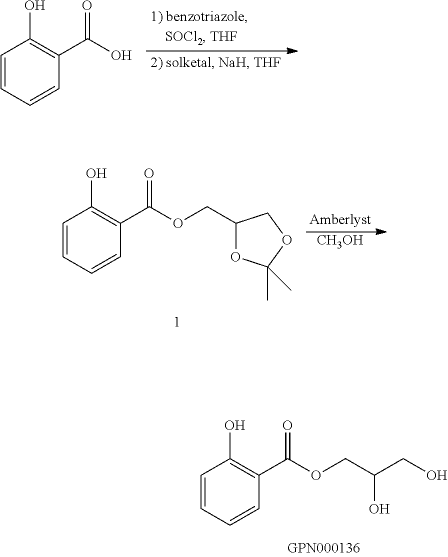

In a flask under argon, 2.17 g (3.15 eq.) of 1H-benzotriazole are dissolved in 30 mL of tetrahydrofuran anhydrous, then 0.42 mL (1 eq.) of thionyl chloride are added. The mixture is stirred for 45 minutes at room temperature then 800 mg (5.792 mmol; 1 eq.) of salicylic acid dissolved in 20 mL of tetrahydrofuran anhydrous are added. The reaction mixture is stirred for 3 hours at room temperature. It is then quickly filtered on sintered. The filtrate is evaporated for obtaining the activated acid (benzotriazol-1-yl(2-hydroxyphenyl)methanone) which is directly involved in the next step.

In a flask under argon, 696 mg (5.266 mmol) of solketal are dissolved in 10 mL of tetrahydrofuran anhydrous and 232 mg (1.1 eq.) of sodium hydride (60% in oil) are added. The mixture is stirred for 30 minutes at room temperature. Then 1.1 eq. (5.792 mmol) of benzotriazol-1-yl(2-hydroxyphenyl)methanone in solution in 20 mL of tetrahydrofuran anhydrous are added. The reaction mixture is stirred for 5 hours at room temperature, then hydrolyzed with 30 mL of water. It is extracted with dichloromethane (3 times 30 mL). The combined organic phases are dried on magnesium sulfate, filtered and evaporated under reduced pressure. The crude is purified by silica gel chromatography (petroleum ether/ethyl acetate 9/1) for obtaining 1.16 g (yield: 87%) of the expected product coded 1 in a form of a colorless oil.

RMN ¹H (250 MHz, CDCl₃): δ 1.39 (s, 3H, CH₃); 1.46 (s, 3H, CH₃); 3.89 (dd, 1H, J=5.3 et 8.7 Hz, CH₂); 4.16 (dd, 1H, J=6.2 and 8.7 Hz, CH₂); 4.33-4.51 (m, 3H, CH₂ and CH); 6.89 (td, 1H, J=1.2 and 8.0 Hz, CH Ar); 6.99 (dd, 1H, J=0.9 and 8.3 Hz, CH Ar); 7.47 (td, 1H, J=1.6 and 8.3 Hz, CH Ar); 7.87 (dd, 1H, J=1.6 and 8.0 Hz, CH Ar); 10.62 (s, 1H, OH).

In a flask, 1 g (3.9640 mmol) of the compound coded 1 are dissolved in 25 mL of methanol and 1.2 g of activated Amberlyst resin 15 are added. The mixture is stirred for 19 hours at room temperature, filtered on sintered. The resin is rinsed with methanol. The filtrate is then evaporated under reduced pressure. The crude is purified by silica gel chromatography (petroleum ether/ethyl acetate: 5/5 then 3/7) for obtaining 620 mg of the compound coded GPN000136 (yield: 74%) in a form of a white solid.

RMN ¹H (250 MHz, CDCl₃): δ 2.61 (si, 1H, OH); 3.07 (Si, 1H, OH); 3.68-3.78 (m, 2H, CH₂); 4.05-4.15 (m, 1H, CH); 4.39-4.51 (m, 2H, CH₂); 6.88 (td, 1H, J=1.0 and 7.6 Hz, CH Ar); 6.98 (dd, 1H, J=0.9 and 8.4 Hz, CH Ar); 7.46 (td, 1H, J=1.7 and 7.7 Hz, CH Ar); 7.84 (dd, 1H, J=1.7 and 8.0 Hz, CH Ar); 10.57 (s, 1H, OH).

RMN ¹³C (62.5 MHz, CDCl₃): 63.5 (CH₂); 65.6 (CH₂); 70.2 (CH); 112.1 (Cq); 117.9 (CH); 119.4 (CH); 130.0 (CH); 136.2 (CH); 161.8 (Cq); 170.3 (Cq).

### EXAMPLE 2: Synthesis of (S)-2,3-dihydroxypropyl 2-hydroxybenzoate (Coded GPS008506)

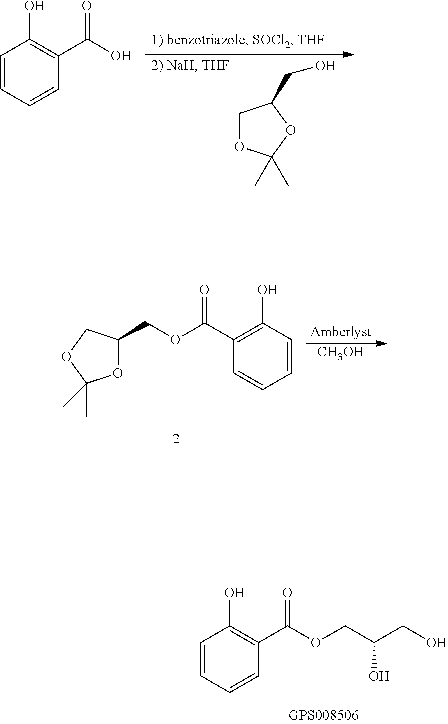

In a flask under argon, 0.3 mg (2.4194 mmol) of (R)-2,3-O-isopropylidene glycerol are dissolved in 5 mL of tetrahydrofuran anhydrous and 106 mg (1.1 eq.) of sodium hydride (60% in oil) are added. The mixture is stirred for 30 minutes at room temperature. Then 637 mg (1.1 eq.) of activated acid described above (benzotriazol-1-yl(2-hydroxyphenyl)methanone) dissolved in 12 mL of tetrahydrofuran anhydrous are added. The reaction mixture is stirred for 4 hours at room temperature then hydrolyzed with 40 mL of water. It is extracted with dichloromethane (3 times 30 mL). The combined organic phases are dried on magnesium sulfate, filtered and evaporated under reduced pressure. The crude is purified by silica gel chromatography (petroleum ether/ethyl acetate 9/1) for obtaining 520 mg (yield: 85%) of the expected product (coded 2) in a form of a colorless oil.

RMN ¹H (250 MHz, CDCl₃): δ 1.39 (s, 3H, CH₃); 1.46 (s, 3H, CH₃); 3.89 (dd, 1H, J=5.3 and 8.7 Hz, CH₂); 4.16 (dd, 1H, J=6.2 and 8.7 Hz, CH₂); 4.33-4.51 (m, 3H, CH₂ and CH); 6.89 (td, 1H, J=1.2 and 8.0 Hz, CH Ar); 6.99 (dd, 1H, J=0.9 and 8.3 Hz, CH Ar); 7.47 (td, 1H, J=1.6 and 8.3 Hz, CH Ar); 7.87 (dd, 1H, J=1.6 and 8.0 Hz, CH Ar); 10.62 (s, 1H, OH).

In a flask, 650 mg (2.5766 mmol) of the compound coded 2 are dissolved in 10 mL of methanol and 800 mg of activated Amberlyst resin 15 are added. The mixture is stirred for 24 hours at room temperature, filtered on sintered. The resin is rinsed with methanol. The filtrate is then evaporated under reduced pressure. The crude is purified by silica gel chromatography (petroleum ether/ethyl acetate: 6/4, 4/6 then 3/7) for obtaining 440 mg of the referenced compound GPS008506 (yield: 80%) in a form of a white solid. Rotating power: α_{D}=+15.5 (c=1; EtOH).

RMN ¹H (250 MHz, CDCl₃): δ 2.61 (sl, 1H, OH); 3.07 (sl, 2H, OH); 3.68-3.78 (m, 2H, CH₂); 4.05-4.15 (m, 1H, CH); 4.39-4.51 (m, 2H, CH₂); 6.88 (td, 1H, J=1.0 and 7.6 Hz, CH Ar); 6.98 (dd, 1H, J=0.9 and 8.4 Hz, CH Ar); 7.46 (td, 1H, J=1.7 and 7.7 Hz, CH Ar); 7.84 (dd, 1H, J=1.7 and 8.0 Hz, CH Ar); 10.57 (s, 1H, OH).

RMN ¹³C (62.5 MHz, CDCl₃): δ 63.5 (CH₂); 65.6 (CH₂); 70.2 (CH); 112.1 (Cq); 117.9 (CH); 119.4 (CH); 130.0 (CH); 136.2 (CH); 161.8 (Cq); 170.3 (Cq).

### EXAMPLE 3

### Synthesis of (R)-2,3-dihydroxypropyl 2-hydroxybenzoate (Coded GPS008505)

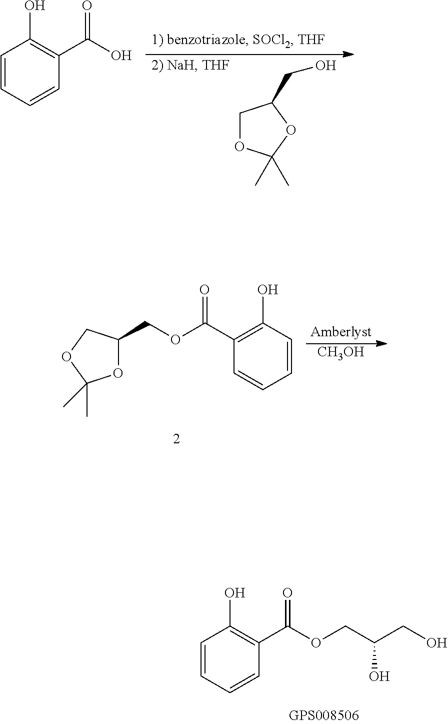

In a flask under argon, 0.4 mg (3.2385 mmol) of (S)-2,3-O-isopropylidene glycerol are dissolved in 7 mL of tetrahydrofuran anhydrous and 142 mg (1.1 eq.) of sodium hydride (60% in oil) are added. The mixture is stirred for 30 minutes at room temperature. Then 852 mg (1.1 eq.) of activated acid described above (benzotriazol-1-yl(2-hydroxyphenyl) methanone) dissolved in 16 mL of tetrahydrofuran anhydrous are added. The reaction mixture is stirred for 5 hours at room temperature then hydrolyzed with 40 mL of water. It is extracted with dichloromethane (3 times 30 mL). The combined organic phases are dried on magnesium sulfate, filtered and evaporated under reduced pressure. The crude is purified by silica gel chromatography (petroleum ether/ethyl acetate 9/1) for obtaining 730 mg (yield: 89%) of the expected product (coded 3) in a form of a colorless oil.

RMN ¹H (250 MHz, CDCl₃): δ 1.39 (s, 3H, CH₃); 1.46 (s, 3H, CH₃); 3.89 (dd, 1H, J=5.3 and 8.7 Hz, CH₂); 4.16 (dd, 1H, J=6.2 and 8.7 Hz, CH₂); 4.33-4.51 (m, 3H, CH₂ and CH); 6.89 (td, 1H, J=1.2 and 8.0 Hz, CH Ar); 6.99 (dd, 1H, J=0.9 and 8.3 Hz, CH Ar); 7.47 (td, 1H, J=1.6 and 8.3 Hz, CH Ar); 7.87 (dd, 1H, J=1.6 and 8.0 Hz, CH Ar); 10.62 (s, 1H, OH).

In a flask, 650 mg (2.5766 mmol) of the compound coded 3 are dissolved in 15 mL of methanol and 800 mg of activated Amberlyst resin 15 are added. The mixture is stirred for 24 hours at room temperature, filtered on sintered. The resin is rinsed with methanol. The filtrate is then evaporated under reduced pressure. The crude is purified by silica gel chromatography (petroleum ether/ethyl acetate: 5/5 then 3/5) for obtaining 480 mg of the referenced compound GPS008505 (yield: 87%) in a form of a white solid. Rotating power: α_{D}=−16.6 (c=1; EtOH).

RMN ¹H (250 MHz, CDCl₃): δ 2.61 (sl, 1H, OH); 3.07 (sl, 1H, OH); 3.68-3.78 (m, 2H, CH₂); 4.05-4.15 (m, 1H, CH); 4.39-4.51 (m, 2H, CH₂); 6.88 (td, 1H, J=1.0 and 7.6 Hz, CH Ar); 6.98 (dd, 1H, J=0.9 and 8.4 Hz, CH Ar); 7.46 (td, 1H, J=1.7 and 7.7 Hz, CH Ar); 7.84 (dd, 1H, J=1.7 and 8.0 Hz, CH Ar); 10.57 (s, 1H, OH).

RMN ¹³C (62.5 MHz, CDCl₃): δ 63.5 (CH₂); 65.6 (CH₂); 70.2 (CH); 112.1 (Cq); 117.9 (CH); 119.4 (CH); 130.0 (CH); 136.2 (CH); 161.8 (Cq); 170.3 (Cq).

Example 4 shows the technical protocol allowing to record the effects of the products according to the invention on chemokines.

### EXAMPLE 4

### Inhibitory Properties of the Derivatives of Formula (I) on Chemokines.

### Cells Expressing the CCR4 Receptor of Chemokines

In order to enable an easy recording of the responses associated to the CCR4 receptor, the cDNA which allows its expression is cloned in the plasmid pIRES (ClonTech) in fusion with the GFP protein, according to Vollmer et al. (1999), which enables to easily measure the expression of the chimeric EGFP-CCR4 receptor. HEK 293 cells (ATCC) are cultured in minimal essential medium MEM (Invitrogen) in the presence of 10% of fetal calf serum (Gibco-BRL), 100 U/mL of penicillin (Invitrogen), 100 αg/mL of streptomycin (Invitrogen) and 2 mM of L-glutamine (Invitrogen) at 37° C. in water-saturated atmosphere and containing 5% of 002. The cells are transfected according to the calcium phosphate precipitate method of Chen ant Okayama, 1986, and the cells having incorporated the expression plasmid are selected with 600 αg/mL of geneticin G-418 (PAA) for 5 weeks. The clones are selected after having been characterized by fluorescence microscopy and cytometric analysis by FACS (fluorescence assisted cell sorting).

### Construction of the Recombinant Protein Gqi5

In order to enable an easy recording of the cell responses associated to the CCR4 receptor, we modify its natural coupling to the inhibition of AMPc production to a coupling to the phospholipase C which enables calcium measurements. We produce for this purpose the chimeric protein Gqi5 by directed mutagenesis of the 5 las amino acids of Gq replaced by their homologues of Gi. The cells expressing the CCR4 receptor are transfected with the plasmid pCNA3.1 in which the cDNA encoding Gqi5 has been incorporated. The recordings of calcium responses are performed the day after de transfection.

### Recording of Cell Calcium Responses and Their Inhibition by the Molecules of the GPN Collection:

The cells are loaded with a fluorescent calcium indicator, INDO-1, according to the protocol provided by the provider (Molecular probes), distributed in microplate wells and placed in a fluorescence reader-pipettor (FlexStation, Molecular devices).

Intracellular calcium elevation is measured by fluorescence intensity change at 401 and 475 nm (excitation 355 nm). Measure points are recorded every 5 s for 150 s for each well. The molecules are tested in the presence of 5 nM of chemokine CCL22 (commercial).

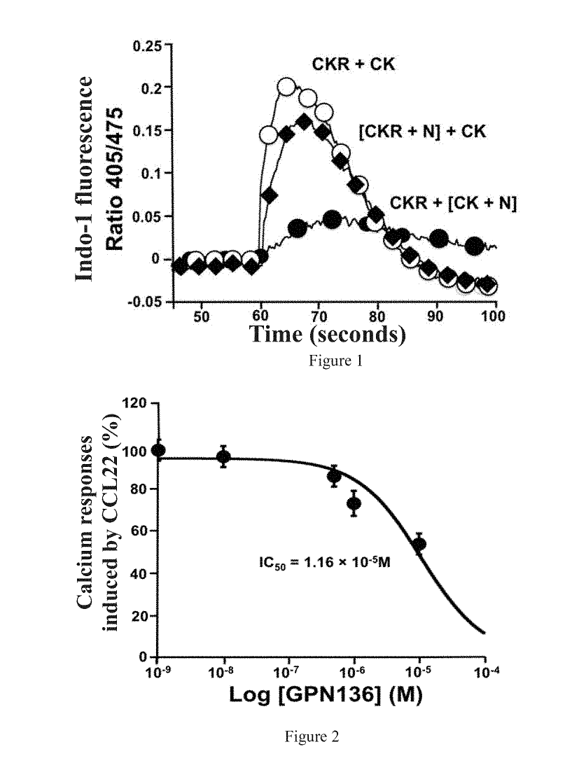

FIG. 1 shows in a general way the obtained results: the addition of chemokine (CK) to cells expressing the chemokine receptor (CKR) leads to a release of intracellular calcium revealed by the fluorescence change of the calcium probe Indo-1. The response develops in a few seconds and fades with time for reaching the return in approximately 2 minutes for a chemokine CCL22 concentration of 5 to 10 nM in HEK 293 cells expressing CCR4. If the molecules to be tested are preincubated with chemokine [CK+N] and if the mixture is added to the cells, we observe a strong decrease in the amplitude of the response if the molecule to be tested is a neutralizing ligand of the chemokine. Conversely, when the neutralizing ligand is preincubated with the cells expressing the receptor [CKR+N] we observe a much smaller decrease of the amplitude of the response.

This protocol has been used for identifying the neutralizing ligands of CCL22 presented in table 1.

The apparent affinity of the GPN136 molecules is determined by the establishment of a dose-effect relationship of the inhibition of the calcium response as presented in FIG. 2 and the results obtained are reported in table 1.

### Table 1: Structure/Function Relationships of the Derivatives of Formula (I)

TABLE 1

| | | Inhibition of | Inhibition of |
|---|---|---|---|
| | | calcium | calcium |
| | | responses | responses |
| | | induced by | induced by |
| Reference | Structure | CCL17 (%) | CCL22 (%) |
| GPN136 | 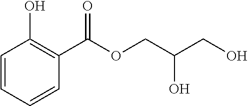 [SMILES: O=C(OCC(O)CO)c1ccccc1O] | 0 | 35 ± 8 |
| GPS008504 | 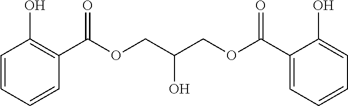 [SMILES: O=C(OCC(O)COC(=O)c1ccccc1O)c1ccccc1O] | 0 | 10 ± 5 |
| GPS008505 | 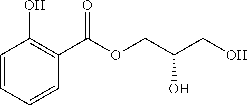 [SMILES: O=C(OC[C@@H](O)CO)c1ccccc1O] | 0 | 37 ± 6 |
| GPS008506 | 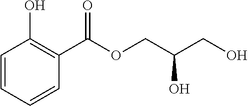 [SMILES: O=C(OC[C@H](O)CO)c1ccccc1O] | 0 | 40 ± 5 |
| GPS008507 | 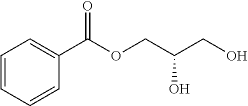 [SMILES: O=C(OC[C@@H](O)CO)c1ccccc1] | 0 | 0 |
| GPS008508 | 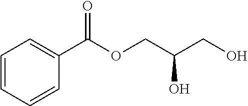 [SMILES: O=C(OC[C@H](O)CO)c1ccccc1] | 0 | 0 |
| Acetylsalicylic acid | 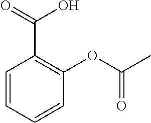 | 0 | 0 |
| GPN0001207 | 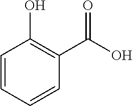 | 0 | 0 |

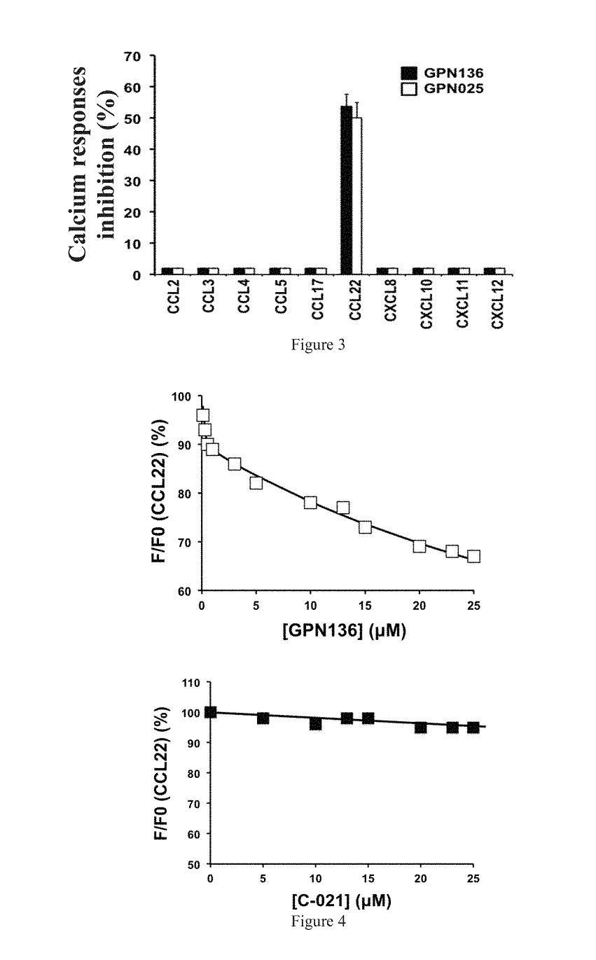

Analysis of the molecules selectivity is carried out by measuring the inhibition of the response evoked by other chemokines on their respective receptors thus, in FIG. 3, the molecules have been tested on the pairs CCL2/CCR2, CCL3/CCR5, CCL17/CCR4, CCL22/CCR4, CXCL8/CXCR8, CXCL10/CXCR2, CXCL11/CXCR3, CXCL12/CXCR4.

FIG. 3 shows the selectivity of the neutralizing ligands of CCL22 (GPN136 (black bars) and GPN025 (white bars)) on the indicated chemokines and their respective receptors.

### Determination of the Interaction Affinity of the Neutralizing Ligands of CCL22 by Fluorescence Extinction of Tryptophan Residues.

This measurement is carried out as described in Hachet-Haas et al. (Small neutralizing molecules to inhibit actions of the chemokine CXCL12, J Biol Chem, 2008, 283(34), 23189-99). Briefly: the fluorescence measurements are carried out on a spectrofluorimeter Fluorolog 3 (JobinYvon/Spex) in a quartz cuvette. CCL22 (1.5 μm (HEPES buffer without albumin) in 1 mL final volume) is excited at 285 nm and its emission spectrum is recorded between 300 and 400 nm after each addition of an aliquot of molecule to be tested (1 μm). The measurements are carried out at 20° C. and the solutions are stirred using a magnetic stir bar. The recorded emission spectra are corrected by subtraction of the emission spectrum of the compound to be tested alone.

FIG. 4 shows that a fluorescence extinction of CCL22 is observed with the neutralizing ligand (GPN 136) only and not with the receptor's agonist (C-021). The shape of the titration curve of CCL22 (1.5 μm) by GPN 136 is biphasic, showing the plausible presence of several binding sites for GPN 136, at least one of which is of high affinity. The adjustment of the traces by the equation (RL)²+(RL)×(−Ro−Lo−KD)+Ro×Lo=0 with (RL) ((Ro+Lo+KD)±((−Ro−Lo−KD)²−4×Ro×Lo)^{1/2})/2 enables to establish the affinity for each site: KD<50 nM for the site of high affinity and KD=53 μm.

There is no signal for the C-021 molecule.

FIG. 4 shows the inhibition of the intrinsic fluorescence of CCL22 by the neutralizing ligand GPN 136 (white squares). The antagonist of the CCR4 receptor, C-021, does not present a decrease of tryptophan fluorescence (black squares).

Example 5 allows showing how the noncytotoxic active candidates are selected.

### EXAMPLE 5

### Properties of the Derivatives on Keratinocytes Migration and Cytotoxicity

The activity of the GPN 136 molecule has been evaluated in a human keratinocytes migration assay performed according to an in vitro wound-healing test in a cell monolayer. It is actually known that, in these chronic pathologies, an important cell migration may promote de formation of pruritus, one of the signs of dermatosis. The immortalized human keratinocytes obtained from the DKFZ (Heidelberg, Germany) are cultivated in Dulbecco medium modified by Eagle (DMEM, Invitrogen) supplemented with 10% of fetal calf serum (Gibco-BRL) and 100 U/ml of penicillin (Invitrogen), 100 μg/ml of streptomycin (Invitrogen) and 2 mM of L glutamine (Invitrogen) at 37° C. and 5% of CO2. The wells of a culture plate of 24 well are inoculated with 200,000 cells and the culture is maintained until formation of a confluent cell monolayer. The cell monolayer is then damaged by performing a linear gap (scratch) using a sterile pipette head of 0.1-10 μL. The medium is then replaced for eliminating the cells and their debris and the culture goes on in different experimental conditions: (i) without any addition, (ii) addition of 100 ng/ml of CCL22 and (iii) addition of a mixture containing 100 ng/ml of CCL22 and the molecule to be tested, for example GPN 136. The effect of the molecules alone is also recorded in different wells. In order to block cell proliferation, each well receives a dose of 1 μg/ml of mitomycin (Sigma-Aldrich).

The culture plates are arranged in the IncuCyte apparatus which records the growth on the cell monolayer by transmitted imaging in cell culture conditions (37° C., 5% CO2, water-saturated atmosphere) for 72 h. Phase-contrast images are collected every 2 hours. Healing is defined (quantified) as the ratio of the width of the scar at time t and time 0. The experiment is repeated three times per condition. The obtained results are reported in FIG. 5.

FIG. 5 shows the migration of human keratinocytes under the effect of GNP136; it shows pictures of monolayers of human Hacat cells after damage at time 0 and after 72 hours of culture in the presence of excipient alone (DMSO, table 1), CCL22 and excipient (CCL22+DMSO, table 2) and CCL22 and GPN136 (CCL22+GPN136, table 3).

The figure shows that cell migration is greatly accelerated by the chemokine CCL22 which acts on the CCR4 receptors present in Hacat cells. This effect of the chemokine CCL22 is abolished by the GPN136 molecule. Some compounds are cytotoxic: for example, the GPN025 molecule.

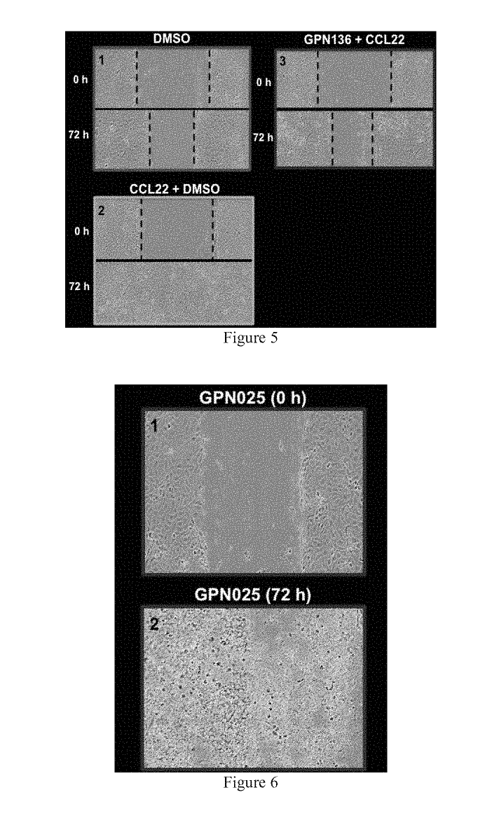

FIG. 6 shows the effect of the GPN025 molecule added on day D0 after damage of the Hacat cell monolayer. It is noted that the cells seem to have formed syncitia after 72 hours of incubation. It is impossible to distinguish the contours. The molecule GPN025 damages the Hacat cells. This effect is quantified in the experiment of metabolic activity presented in FIG. 6.

The metabolic activity test is a colorimetric test (WST-1 assay—Ozyme) based on the measurement of the activity of the mitochondrial enzyme succinate-tetrazolium reductase cleaving the tetrazolium salt WST-1 ((4-[3-(4-Iodophenyl)-2-(4-nitrophenyl)-2H-5-tetrazolio]-1,3 benzene disulfonate)) in a water-soluble derivative called formazan, a dye which absorbs light in the range of 420-480 nm. Hacat cells are distributed at the rate of 200,000 cells per well in a culture plate of 24 wells, in a final volume of 500 μL. They are cultured for 24 hours before adding mitomycin C (1 μg/ml) and the compound to be tested. The culture plate is incubated for 72 hours at 37° C., 5% CO2, in water-saturated atmosphere. The medium is then replaced by fresh medium supplemented with reagent WSR-1 then incubated for 2 hours at 37° C. The plates are stirred to homogenize the color in the wells and the absorbance is measured at 450 nm in the multiwell-plate reader. The measurements are carried out in triplicates. As enzymatic activity requires cellular integrity, the absorbance will be all the stronger as the metabolic state of the cells is good.

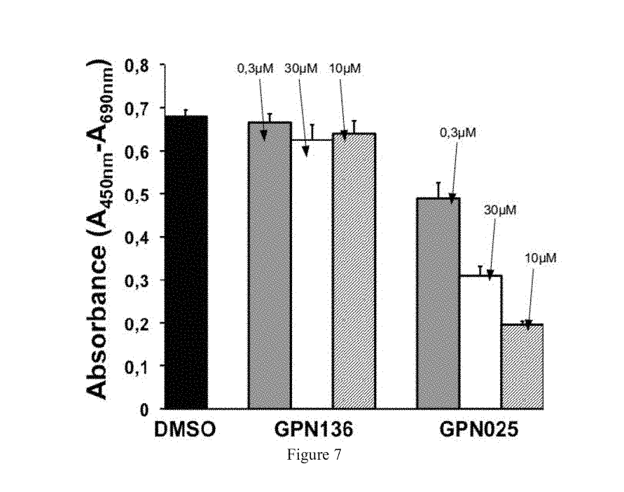

FIG. 7 shows the cytotoxic activity of GPN136 and GPN025.

In vitro cytotoxic activity: the Hacat cell cultures are treated with different concentrations of molecule: 0.3 μM (dark gray bars), 10 μM (light gray bars) or 30 μM (white bars) of GPN 136 and GPN 025 molecules. The activity of the mitochondrial enzyme formazan-reductase is recorded using the chromogenic substrate WST-1 which turns into a colored product, called formazan, detected at 450 nm. As shown in FIG. 7, the GPN 136 molecule does not affect enzymatic activity. In contrast, the GPN 025 molecule reduces in a dose-dependent manner and significantly the cellular metabolism, thus revealing a cytotoxic effect of GPN 025.

### EXAMPLE 6

This example presents a formulation of the composition according to the invention.

| COMMERCIAL | | PERCENTAGE IN |
|---|---|---|
| NAME | INCI NAME | THE FORMULA |
| Simulsol 165 | PEG-100 Stearate/ | 3 |
| | Glyceryl Stearate | |
| Montanov L | C14-22 Alcohol/C12- | 2 |
| | 20 Alkyl Glucoside | |
| Safflower oil | Carthamus Tinctorius | 2 |
| | Seed Oil | |
| Shea butter | Butyrospermum Parkii | 4 |
| | Butter | |
| Ceramidone | Octyldodecyl PCA | 3 |
| Phytosqualane | Squalane | 5 |
| DUB MCT 5545 | Caprylic/Capric | 7 |
| | Triglycerides | |
| Beeswax | Cera Alba | 1 |
| Sorbic acid | Sorbic acid | 0.1 |
| Glycerin | Glycerin | 2 |
| Sorbitol | Sorbitol | 3 |
| Aristoflex AVC | Ammonium | 1.3 |
| | Acryloyldimethyltaurate/ | |
| | VP copolymer | |
| Xanthan gum | Xanthan gum | 0.2 |
| Nicotinamide | Niacinamide | 1 |
| Lipacide C8G | Capryloyl glycine | 0.5 |
| Purified water | Aqua | Qsp to 100 |
| GPN136 | — | 3 |

This composition is applied every day for one month to the skin of the face of a 20-year-old man suffering from atopic dermatitis over the whole face; this application consists in an application of 2 g/day for the whole face. A total progressive disappearance of the zones of inflammation is observed at the end of the treatment.

Ingredients which may be combined with the compositions for topic application in order to enhance the effect or reach action synergies have been proposed below, in particular with the formulation of the present example 6 (the amounts are given in percentage by weight):

a) ingredients having anti-inflammatory properties commonly used in cosmetic products, in particular nicotinamide (1 to 4%), 18 beta-glycyrrhetinic acid (0.01 to 1%), alpha bisabolol (0.1 to 1%);
b) glycine having a soothing effect (1 to 3%);
c) silver micro-particles (0.1%) which enable to regulate, stabilize the microbial flora and avoid superinfection;
d) an anti bacterial-adhesion agent for limiting adhesion and proliferation of Staphylococcus aureus such as Teflose® by Solabia group composed of propanediol, rhamnose, glucose and glucuronic acid (2 to 4%);
e) the unsaponifiables of shea butter and sunflower for strengthening the skin barrier and improving skin hydration (0.5 to 2%);
f) a chicory extract of 1 to 3% (such as Vederine® by Silab) for the reinforcement and recovery of the skin barrier;
g) a raspberry seed oil and vitamin E succinate derivative (Raspberry seed oil/Tocopheryl succinate/Aminopropanediol esters) at 1% such as Vitaskin® E by Solabia;
h) octyldodecyl PCA at 0.5 to 5% for stimulating the synthesis of epidermal lipids;
i) a safflower vegetable oil or stabilized evening primrose in a ceramide-like form such as “Omega 6 Ceramide® Safflower” and “Omega 6 Ceramide® Evening primrose” by Solabia for improving cell cohesion (0.1 to 1%)
j) an optimized mixture of argan oil, shea butter and barley wax, as in “Stimutex® AS” by DSM, for an anti-inflammatory action and for decreasing histamine release (2 to 5%);
k) a mixture of white birch bark and Scrophularia nodosa extract, as in Protectol® by Greetech, for its anti-inflammatory properties due to the presence of betulinic acid (1 and 3%);
l) a mixture of lactose and milk protein at 0.5% as in Modukine™ by CLR for its anti-inflammatory properties.

1-17. (canceled)

18. A method for treating inflammatory diseases of the human or animal skin in an individual, comprising administering to the individual a dermocosmetic or pharmaceutical composition comprising, in an acceptable vehicle, an effective amount of at least one active agent constituted by a compound having the following general formula (I): 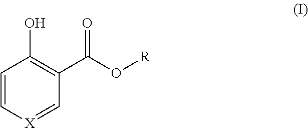
wherein:
X represents a —CH— group or a nitrogen atom;
R represents a (C₁-C₆) alkyl, a (C₃-C₆) cycloalkyl group, wherein one or more —CH₂— groups may be replaced by —O— or may be substituted by one or more radicals selected from (C1-C6) alkyl, hydroxy or alkoxy radicals, said compound of formula (I) may be in the form of a basic or acid addition salt.

19. The method of claim 18, wherein the alkyl radical(s) is (are) selected from the group consisting of methyl, ethyl, propyl, isopropyl, butyl, isobutyl, tert-butyl, pentyl, neopentyl, and n-hexyl radicals substituted or not by one or more hydroxyl groups or by one or more alkoxy groups.

20. The method of claim 18 wherein R is a mono or polycyclic cycloalkyl radical.

21. The method of claim 18, wherein, when the implemented composition contains at least one compound of formula (I) comprising at least one alkoxy radical, said alkoxy radical is selected from the group consisting in methoxy, ethoxy, n-propyloxy, i-propyloxy, n-butoxy, s-butoxy, t-butoxy, n-pentoxy and s-pentoxy groups, each alkoxy group may be substituted by an alkyl radical selected from the group consisting of methyl, ethyl, propyl, isopropyl, butyl, isobutyl, tert-butyl, pentyl, neopentyl and n-hexyl radicals substituted or not by one or more hydroxyl groups or by one or more alkoxy groups.

22. The method of claim 18, wherein the active agent is an acid addition salt, said salt being selected from the group consisting of hydrochloric acid, hydrobromic acid, sulfuric acid, phosphoric acid, acetic acid, trifluoroacetic acid, lactic acid, pyruvic acid, malonic acid, succinic acid, glutaric acid, fumaric acid, tartric acid, maleic acid, citric acid, ascorbic acid, methane- or ethane-sulfonic acid and camphoric acid.

23. The method of claim 18, wherein the active agent is a basic addition salt, said base being selected from the group consisting of sodium or potassium hydroxide, triethylamine and tert-butylamine.

24. The method of claim 18, wherein the composition comprises at least one active agent constituted by a compound of general formula (I) selected from the group consisting of
2,3-dihydroxypropyl 2-hydroxybenzoate,
(S)-2,3 dihydroxypropyl 2-hydroxybenzoate,
(R)-2,3-dihydroxypropyl 2-hydroxybenzoate,
2,3-dihydroxypropryl, 4-hydroxynicotinate and its isomers,
dipropylene glycol salicylate and its isomers, and
propylen glycol salicylate and its isomers.

25. The method of claim 18, wherein the composition comprises from 0.10% to 5% by weight of active agent(s) of formula (I) with respect to the total weight of the composition.

26. The method of claim 18, wherein the active agent(s) of formula (I) is (are) in the form of a powder, is (are) adsorbed on powdery organic polymers, or is (are) in an encapsulated form.

27. The method claim 26, wherein, when an active agent of formula (I) of the implemented composition is (are) in an encapsulated form, its encapsulation means is (are) selected from the group consisting of microspheres, liposomes, glycospheres, chylomicrons, macro-, micro and nanoparticles, macro-, micro- and nanocapsules.

28. The method of claim 18, wherein the composition comprises at least one complementary active agent different from a compound of formula (I), said complementary agent being selected from the group consisting of sun-protection agents, anti-wrinkles agents with antiradical, antioxidant or anti-irritant activity, agents promoting cellular nutrition, respiration, hydration or regeneration, anti-seborrheic agents, agents promoting skin tonicity, hair protection agents, cicatrizing agents, hyaluronic acid, amino acids, anti-aging agents and after-sun agents.

29. The method of claim 18, wherein the composition is formulated in a form selected from the group consisting of lotion, gel, emulsion, cream or milk, oil in water or water in oil biphasic emulsion, triphasic emulsion, body oil, shampoo, soap, mask, ointment, stick and pencil for makeup, lip protective stick, nanocapsules, liposomes and transdermic patch for topic applications.

30. The method according to claim 18, wherein the composition comprises at least one product selected from the group consisting of mucopolysaccharides, vitamins, ceramides, vegetal oils and agents effective in dermatoses.

31. The method of claim 18 wherein the composition comprises at least one product selected from the group consisting of antibacterial agents, perfumes, extraction and/or synthesis lipids, gelling and viscosity polymers, surfactants, emulsifiers, plant extracts, tissue extracts, marine extracts, hydro- or lipo-soluble active substances, and synthesis active substances.

32. The method of claim 18 wherein the composition is administrable by the topical or oral route.

33. The method of claim 18, for treating a disease selected from the group consisting of dermatoses, acne, atopic dermatitis, psoriasis, eczema, dry skin with atopic tendencies, red skin, Crohn's disease, asthma, allergic asthma and allergic rhinitis.

34. The method of claim 18, for treating dermatological diseases linked to seborrheic, acne, inflammatory and immunological activities.

35. A method for inhibiting at least one chemokine of the human or animal skin in an individual, comprising administering to the individual a dermocosmetic of pharmaceutical composition comprising, in an acceptable vehicle, an effective amount of at least one active agent constituted by a compound having the following general formula (I): 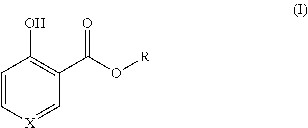
wherein:
X represents a —CH— group or a nitrogen atom;
R represents a (C₁-C₆) alkyl, a (C₃-C₆) cycloalkyl group, wherein one or more —CH₂— groups may be replaced by —O— or may be substituted by one or more radicals selected from (C1-C6) alkyl, hydroxy or alkoxy radicals, said compound of formula (I) may be in the form of a basic or acid addition salt.

36. The method of claim 35, wherein the compound of formula (I) is selected from the group consisting of
2,3-dihydroxypropyl 2-hydroxybenzoate,
(S)-2,3 dihydroxypropyl 2-hydroxybenzoate,
(R)-2,3-dihydroxypropyl 2-hydroxybenzoate,
2,3-dihydroxypropryl, 4-hydroxynicotinate and its isomers,
dipropylene glycol salicylate and its isomers, and
propylen glycol salicylate and its isomers.

37. The method of claim 35, wherein the chemokine is CCL22 and/or CCL17
